(19) United States
(12) Patent Application Publication
(10) Pub. No.: US 2022/0061740 A1
(43) Pub. Date: Mar. 3, 2022

(54) SYSTEM AND METHOD FOR CONCUSSIVE IMPACT MONITORING

(71) Applicant: New York University, NEW YORK, NY (US)

(72) Inventor: André A. FENTON, New York, NY (US)

(21) Appl. No.: 17/445,915

(22) Filed: Aug. 25, 2021

Related U.S. Application Data
(60) Provisional application No. 62/706,624, filed on Aug. 28, 2020.

(51) Int. Cl. A61B 5/00; A61B 5/374; A61B 5/11; A61B 5/256; A61B 5/291; G16H 50/70; G16H 40/67; G01P 15/18

(52) U.S. Cl. CPC A61B 5/4076; G16H 50/20; A61B 5/1114; A61B 5/256; A61B 5/291; A61B 5/746; A61B 5/7455; A61B 5/7445; A61B 5/7405; G16H 50/70; G16H 40/67; G01P 15/18; A61B 2562/0219; A61B 2562/0209; A61B 2562/043; A61B 2562/06; A61B 5/374

(57) ABSTRACT

A system and a method for detecting mild traumatic brain injury (mTBI) includes a memory arrangement including stored brain data corresponding to electrical activity of a brain of a subject at locations in the brain during a first time period. In addition, the system includes a processor receiving the stored brain data and current brain data corresponding to electrical activity of the brain of the subject at the plurality of locations during a second time period, wherein the second time period is after the first time period. The processor generates a first set of phase synchrony measures (PSM) corresponding to frequency band-specific oscillatory phase synchrony of the stored brain data, and a second set of PSM corresponding to frequency band-specific oscillatory phase synchrony of the current brain data. The processor determines a likelihood of mTBI based on the first and second sets of PSM. The stored brain data may be a normative distribution of data of the electrical activity of the brain determined from a set of standards

### PRIORITY CLAIM

The present disclosure claims priority to U.S. Provisional Patent Application Serial No. 62/706,624 filed on Aug. 28, 2020, the entire disclosure of which is incorporated herein by reference.

### FIELD

The present disclosure relates to systems and methods for identifying a concussive impact of a patient, and specifically, to systems and methods for monitoring a patient to detect mild traumatic brain injuries and in so doing, make recommendations for consumer good purchases.

### BACKGROUND

Objective tests are not yet available that can identify a Mild Traumatic Brain Injury (mTBI), i.e., a concussion. Magnetic Resonance Imaging (MRI) techniques, e.g., Diffusion Tensor Imaging (DTI) have shown promise in detection of subtle white matter anomalies after mTBI. However, MRI and DTI are expensive, non-portable systems. Furthermore, physiological conditions that underlie mTBI are thought to also change with time, making it unlikely that static measurements (e.g., MRI and DTI) of brain structure can identify mTBI. Consequently, a measure of brain function that is sensitive to mTBI is needed.

### SUMMARY

One of embodiments of the present disclosure provides a system for detecting mild traumatic brain injury (mTBI). The system includes a memory arrangement including stored brain data corresponding to electrical activity of a brain of a subject at a plurality of locations in the brain during a first time period. The system further includes a processor. The processor receives the stored brain data and current brain data corresponding to electrical activity of the brain of the subject at the plurality of locations during a second time period, wherein the second time period is after the first time period. The processor further generates a first set of phase synchrony measures (PSM) corresponding to frequency band-specific oscillatory phase synchrony of the stored brain data, and a second set of PSM corresponding to frequency band-specific oscillatory phase synchrony of the current brain data. The processor further determines a likelihood of mTBI based on the first and second sets of PSM.

In another aspect of the present disclosure, a method for detecting mild traumatic brain injury (mTBI). The method includes accessing stored data corresponding to electrical activity of a brain of a subject at a plurality of locations in the brain during a first time period. The method further includes collecting current data corresponding to electrical activity of the brain of the subject at the plurality of locations during a second time period, wherein the second time period is after the first time period. The method further includes generating a first set of phase synchrony measures (PSM) corresponding to frequency band-specific oscillatory phase synchrony of the stored brain data, and a second set of PSM corresponding to frequency band-specific oscillatory phase synchrony of the current brain data. The method further includes determining a likelihood of mTBI based on the first and second sets of PSM.

These and other aspects of the present disclosure will become apparent to those skilled in the art after a reading of the following detailed description of the present disclosure, including the figures and appended claims.

### BRIEF DESCRIPTION OF DRAWINGS

FIG. 1 shows an exemplary system for a concussive impact monitoring.

FIG. 2 shows a perspective view of a protective head covering of the system of FIG. 1.

FIG. 3 shows a perspective view of a wearable garment of the system of FIG. 1.

FIG. 4 shows a first computing unit of the protective head covering of FIG. 2.

FIG. 5 shows a second computing unit of the wearable garment of FIG. 3.

FIG. 6 shows an exemplary method for utilizing the system of FIG. 1.

FIG. 7 shows another exemplary method for utilizing the system of FIG. 1.

### DETAILED DESCRIPTION OF DRAWINGS

The present disclosure may be further understood with reference to the following description and the appended drawings, wherein like elements are referred to with the same reference numerals.

DTI studies show that mTBI selectively damages associational, commissural and corticothalamic white matter tracts, which are the myelinated long-axon fibers of the brain. The same associational, commissural and corticothalamic tracts are required for long distance interareal synchronization of locally generated neural network oscillations that may be detected in an Electroencephalogram (EEG). During tasks that require the coordinated activation of distant brain regions, these oscillations synchronize into multi-frequency, cross-frequency “braided” or “nested” oscillations between the phase of a slow oscillation and the amplitude of a fast oscillation in the gamma, alpha, theta and delta bands. These “cross-frequency” phase-amplitude synchrony patterns may be observed at single electrodes. Furthermore, a variety of cognitive processes produce long distance EEG phase, amplitude, and cross-frequency “interareal” synchrony patterns that can be measured between electrode pairs within specific frequency bands that identify the biophysical origin of the oscillation. Mental task-specific patterns of cross-frequency and interareal synchronies may be detected in the scalp EEG during performance of specific neuropsychological tests.

The systems and methods of the present application provides a way to detect and/or predict whether or not patients are experiencing mTBI, by detecting subtle injuries to their long white matter tracts. Such injuries may be detected by identifying abnormal cross-frequency and/or interareal synchronies in their scalp EEG. The systems and methods described herein may also identify traumatic insults to the head that produce especially strong shearing forces that are likely to cause the white matter injuries that manifest as mTBI. The ability to classify resting state EEGs from patients (e.g., athletes) as either baseline (pre-injury) or post-injury may have at least 80% accuracy. This classification can be largely based on measures of long-range synchrony in the EEG, specifically a measure of phase synchrony known as Phase-Locking Value (PLV).

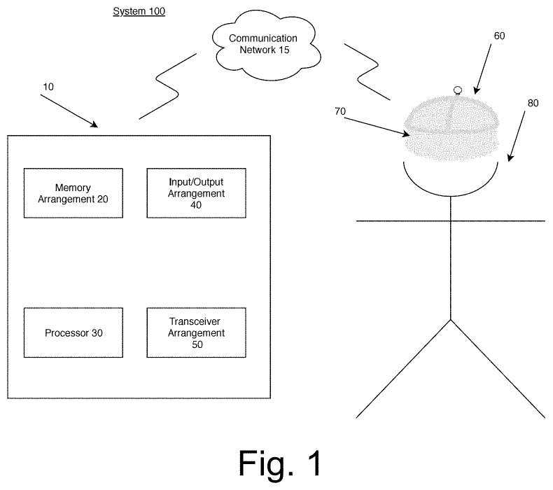

FIGS. 1-5 show an exemplary embodiment of a system 100 for concussive impact monitoring and detection of an mTBI in a subject. The system 100 may include a computing device 10, a protective head covering 60 and a wearable garment 70. The wearable garment 70 is placed onto a head of a subject 80. The head covering 60 is placed onto the wearable garment 70, such as, for example, as an inner lining to the head covering 60. The computing device 10, the head covering 60 and the wearable garment 70 may communicate with one another via a communications network 15.

In the exemplary embodiment, the computing device 10 may be a portable and/or a handheld device (e.g., a phone, a tablet, a laptop, etc.). In another embodiment, the computing device 10 may be a stationary device (e.g., a medical cart with a computer). The computing device 10 may include a memory arrangement 20, a processor 30, an input/output arrangement 40 and a data communications device. The memory arrangement 20 stores a plurality of data, including data corresponding to electrical activity of a brain of the subject 80. In an exemplary embodiment, the data communications device is a transceiver arrangement 50 that may communicate with the head covering 60 and the wearable garment 70 over the communications network 15 to receive and transmit data using a plurality of protocols (e.g., WiFi, Bluetooth). In the exemplary embodiment, the communication network 15 may be a wireless communication network (e.g., WiFi, cell phone network, wireless local area networks, wireless sensor networks, satellite communication networks, terrestrial microwave networks, etc.). The input/output arrangement 40 is any device which may input or output data. The input/output arrangement 40 may be one of a keyboard, a mouse, a touch sensitive display and/or a monitor. Additionally, the input/output arrangement 40 may have a port for connecting the computing device 10 to a further device via a direct connection (e.g., Ethernet cable, USB).

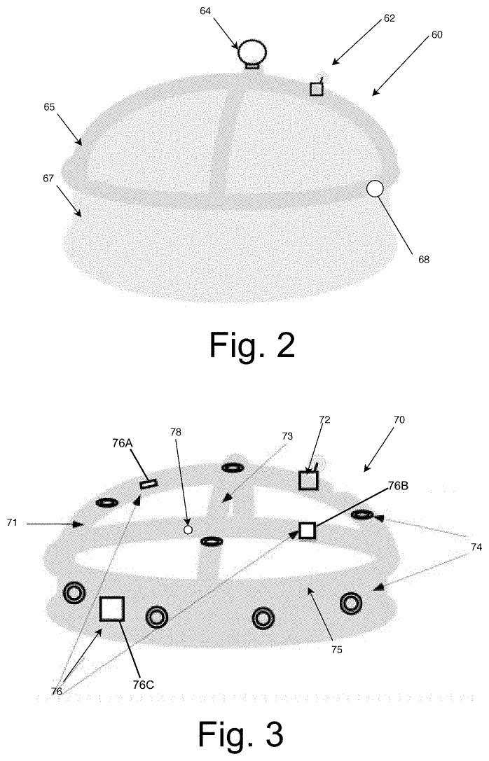

FIG. 2 shows a perspective view of the head covering 60 according to an exemplary embodiment. The head covering 60 may be sized and shaped to conform to a shape of a head of the subject 80. In the exemplary embodiment, the head covering 60 may be a helmet. The head covering 60 may have a first computing unit 62, a status notification device 64, a shell 65, an inner lining 67 and a portable power arrangement 68. The head covering 60 may conform to a top portion of the head of the subject 80 (e.g., a hockey helmet, a baseball helmet, etc.) or a substantial portion (i.e., more than 75%) of the head of the subject 80, (e.g., a football helmet, a motorcycle helmet, etc.). The shell 65 may be an outer shell of a helmet. The shell 65 may be a thin, hard layer which may be composed of at least one of polycarbonate plastic, fiberglass, a Kevlar material, and other material that is capable of absorbing shock. The inner lining 67 is a soft layer which may be composed of expanded polystyrene or another suitable foam or fiber like material. The portable power arrangement 68 provides power to the head covering 60. The portable power arrangement 68 may be a portable battery.

FIG. 4 shows an exemplary embodiment of the first computing unit 62. The first computing unit 62 may include a first memory arrangement 82, a first processor 83, a first input/output arrangement 84, and a first transceiver arrangement 85. The first computing unit 62 may be attached to the head covering 60. The first transceiver arrangement 85 is capable of connecting to the communication network 15 to communicate with the computing device 10. The first memory arrangement 82 stores data and may be, e.g., an SD card, similar to the memory arrangement 20. The first processor 83 processes data that is received by the first transceiver arrangement 85 from the computing device 10. The first input/output arrangement 84 may connect the first computing unit 62 to at least one of the computing device 10 and the wearable garment 70 via a direct connection (e.g., Ethernet cable, USB).

The status notification device 64 is a device that may display an output. The status notification device 64 may be an electrochromic, voltage-controlled color changing paint, a Light

Emitting Diode (LED), a speaker, a transmitter, or any combination of the above. The status notification device 64 is capable of relaying data received by the first transceiver arrangement 85. Moreover, the status notification device 64 may be an external signal that uses at least one of light, sound, or radio transmission to relay the data.

FIG. 3 shows a perspective view of the wearable garment 70 according to an exemplary embodiment. The wearable garment 70 may include a plurality of bands, a second computing unit 72, a plurality of electrodes 74, one or more accelerometers 76, and a portable power arrangement 78. The wearable garment 70 may be any garment that at least partially covers the head of the subject 80, for example, the wearable garment 70 may cover at least ½ of the neurocranium of the subject 80. In the exemplary embodiment, the wearable garment 70 is a headband or an insert into helmet. The wearable garment 70 may be composed of a median coronal band 71, a median sagittal band 73 and a transverse band 75. The transverse band 75 may be a sweatband extending about a radial circumference of the head. Each of the bands 71, 73 and 75 may comprise an absorbent and elastic material (e.g., terrycloth, neoprene, etc.).

The wearable garment 70 may house a plurality of electromechanical devices. Further, the wearable garment 70 may include a plurality of pockets, each of the pockets sized and shaped to store one or more electromechanical devices. In the exemplary embodiment, the electromechanical devices are the plurality of accelerometers 76 which measure acceleration and estimate impact. In particular, the accelerometers 76 may be configured to measure linear acceleration in vertical and/or horizontal planes. The accelerometers 76 may also be configured to measure rotational acceleration in pitch, yaw, and/or roll. The accelerometers 76 may be optimized for estimating shearing forces as opposing planar accelerations. In one of the exemplary embodiments of the wearable garment 70, there are three accelerometers 76. However, one ordinarily skilled in the art will understand that the wearable garment 70 may have a greater or a smaller number of accelerometers 76.

The accelerometers 76 may be situated at a plurality of locations on the head of the subject 80. For example, as shown FIG. 3, accelerometer 76A is placed on the coronal band 71 and accelerometers 76B and 76C are placed on the transverse band 75. The accelerometers 76B and 76C may be spaced such that the accelerometer 76C is on an opposite side of the transverse band 75, relative to the accelerometer 76B. In another embodiment, there may be at least one further accelerometer 76 on each of the bands 71, 73 and 75, physically arranged around the wearable garment 70 (not shown).

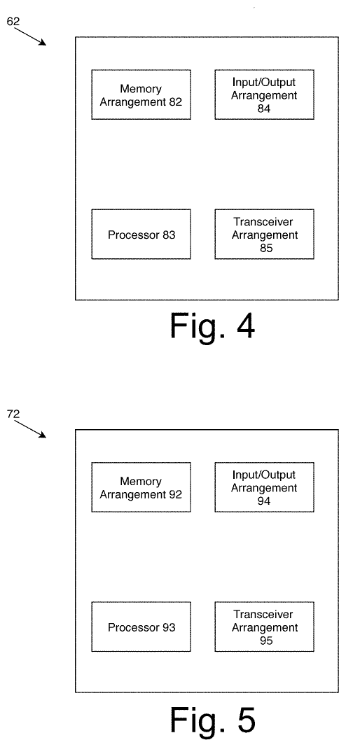

FIG. 5 shows an exemplary embodiment of the second computing unit 72. Similar to the first computing unit 62, the second computing unit 72 may include a second memory arrangement 92, a second processor 93, a second input/output arrangement 94 and a second transceiver arrangement 95. The second computing unit 72 may be attached to the wearable garment 70. The second transceiver arrangement 95 is capable of connecting to the communications network 15 to communicate with the computing device 10. The second memory arrangement 92 stores data and may be, e.g., an SD card, similar to the memory arrangement 20. The second processor 93 processes data that is received by the second transceiver arrangement 95. The second processor 93 processes data that is transmitted by the second transceiver arrangement 95 to the computing device 10. The second input/output arrangement 94 may connect the second computing unit 72 to at least one of the computing device 10 and the head covering 60 via a direct connection (e.g., Ethernet cable, USB). The portable power arrangement 78 provides power to the wearable garment 70. The portable power arrangement 78, similar to the portable power arrangement 68, may be a portable battery. The exemplary embodiment of the system 100 has two computing units 62 and 72, however one with ordinary skill in the art will understand that computing units 62 and 72 as described above may also be implemented as a single computing unit located on either the head covering 60 or the wearable garment 70. Similarly, the exemplary embodiment of the system 100 has two transceiver arrangements 85 and 95, however one with ordinary skill in the art will understand that the transceiver arrangements 85 and 95 as described above may also be implemented as a single transceiver arrangement located on either the head covering 60 or the wearable garment 70.

The electrodes 74, as shown in in FIG. 3 collect data from the brain of the subject 80. Each of the electrodes 74 may be placed into a socket of the wearable garment 70, wherein the socket is sized and shaped to fit the electrode 74. The electrodes 74 within the wearable garment 70 are arranged such that they make a galvanic contact (i.e., direct electrical connection) with a portion of the head of the subject 80 at discrete locations. More specifically, the portion of the head is at least one of forehead skin and scalp of the subject 80. The electrodes 74 detect electrical activity of the brain from the discrete locations. Additionally, the electrodes 74 are couplable to the second processor 93 to transfer data correspond to detected electrical activity thereto. The electrical activity may be converted from analog data to digital data via an analog to digital convertor (not shown) that couples the electrodes 74 to the second processor 93. The digital data may then be transmitted to the computing device 10. In one of the exemplary embodiments of the wearable garment 70, there are sixteen electrodes 74. However, one ordinarily skilled in the art will understand that the wearable garment 70 may have a greater or fewer number of electrodes 74.

In the exemplary embodiment, the wearable garment 70 is a separate device that is placed onto the head of the subject 80 in the desired configuration. The head covering 60 is placed onto the head of the subject 80, at least partially covering the wearable garment 70. In another embodiment, the wearable garment 70 may be attached to the inner lining 67 of the head covering 60. An adhesive method or a stitching method may be used to attach the wearable garment 70 to the inner lining 67. An outcome of attaching the wearable garment 70 to the inner lining 67 is a singular device, i.e., the wearable garment 70 and the head covering 60 form one piece. Thus, the head covering 60 may be placed in the desired configuration onto the head of the subject 80 without first placing the wearable garment 70 in the desired configuration.

The first and second processors 83, 93 may be microprocessors such that each one would fit into size constraints of the respective first and second computing units 62, 72. In more detail, the second processor 93 stores digitized EEG signals from the electrodes 74 on the second memory arrangement 92 along with digitized accelerometer outputs from the plurality of accelerometers 76, for offline analysis. The digitized EEG signals are processed in real-time by the processor 30 to estimate head impact. In parallel, the second processor 93 computes synchrony (e.g., long-range phase synchrony) of selected EEG signals at distant pairs of the electrodes 74. Large impact and consequent changes of synchrony increase the estimated likelihood of concussion according to a method for detecting mTBI (i.e., mTBI-determining method) that will be described below.

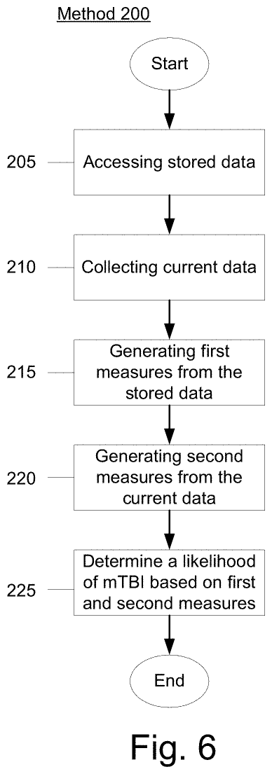

FIG. 6 shows an exemplary method 200 for detecting mTBI of the subject 80 using the system 100. In one of the exemplary embodiments, the method 200 will be applied to the subject 80 who is a football player playing American football. Prior to a beginning of the football game, the wearable garment 70 is placed onto the head of the subject 80 in a predetermined manner. In the predetermined manner, the electrodes 74 are positioned such that the electrodes 74 contact the head at the discrete locations. Next, the subject 80 places the head covering 60, which is a football helmet in the example, onto their head, on top of the wearable garment 70. The method 200 begins once the wearable garment 70 and head covering 60 are placed onto the head of the subject 80.

In step 205, the processor 30 accesses a first set of data (also referred as stored brain data herein) which is stored on the memory arrangement 20. The first set of data is collected during a first period of time (i.e., uninjured time). The first time period corresponds to a period of time during which the subject 80 is relatively inactive while having diverse functioning of an uninjured brain, and/or when it is known that the subject does not suffer from mTBI. In the above-mentioned example, the first time may be prior to a football game. For the first data to be collected, a user (e.g., a coach or a side-line medic, etc.) activates the wearable garment 70 once it is placed onto the head of a player. When the wearable garment 70 is activated, the electrodes 74 and/or the accelerometers 76 are activated. When the electrodes 74 are activated, the electrodes 74 measure electrical activity of the brain of the subject 80. The electrical activity may be EEG signals, such as in the exemplary embodiment. When the accelerometers 76 are activated, the accelerometers 76 measure acceleration motion of the head of the subject 80, for example, motion of the player's head during the course of the football game (e.g., while running, being tackled, hitting the ground, etc.).

Upon collection of the electrical activity by the electrodes 74, the electrical activity is processed in the second processor 93 according to a predetermined protocol. The protocol in the exemplary embodiment, may be converting the electrical activity into a digital format via the analog to digital convertor of the second computing unit 72. The first set of data is transmitted by the second transceiver arrangement 95 to the memory arrangement 20. Similarly, upon collection of the acceleration output by the accelerometers 76, the acceleration output is processed in the second processor 93 according to the predetermined protocol. In summary, data corresponding to the electrical activity of the brain of the subject 80 and/or acceleration motion of the head of the subject 80 are converted into the digital format. The first set of data may comprise data corresponding to the electrical activity of the brain of the subject 80 collected during the first time period. The first set of data may optionally include data corresponding to the acceleration motion of the head of the subject 80 during the first time period.

In step 210, the processor 30 accesses a second set of data (also referred as current brain data herein) and motion data which are both collected during a second time period (i.e., a current time). In an exemplary embodiment, the second time period corresponds to a period of time during which the subject 80 is active, for example when the subject 80 is engaged in a physical activity. The second set of data is collected by the electrodes 74 when the wearable garment 70 is activated at the second time, the second time being after a pre-determined event. The motion data is collected by the accelerometers 76 when the wearable garment 70 is activated at the second time. The pre-determined event may be a two-minute warning, a ten-minute interval, or when the subject 80 increases in activity such that an impact to the head is more likely to occur (e.g., participating in a football game).

In the exemplary embodiment, the processor 30 is able to determine whether the subject 80 encounters an impact to the head. An impact protocol by which the impact is determined will be described below. During the second time period, the electrodes 74 measure electrical activity of the brain of the subject 80 at the plurality of discrete locations. Similar to data corresponding to the electrical activity of the brain collected during the first time period, data corresponding to the electrical activity of the brain collected during the second time period may be converted into a digital format and transmitted to the processor 30 via substantially the same protocol described above. After the second set of data is processed by the processor 30, the second set of data may be stored on the memory arrangement 20. Similarly, upon collection of motion data corresponding to the acceleration motion of the head of the subject 80 by the accelerometers 76, the motion data collected during the second time period is also processed in the second processor 93 in a similar manner as the acceleration data collected during the first time period. The second set of data may comprise data corresponding to the electrical activity of the brain of the subject 80 collected during the second time period. The motion data may include data corresponding to the acceleration motion of the head of the subject 80 during the second time period.

In step 215, the processor 30 generates a first set of frequency-band-specific oscillatory phase synchrony measures (PSM) from the first set of data. In step 220, the processor 30 generates a second set of frequency-band-specific oscillatory PSM from the second set of data. In steps 215 and 220, the processor 30 generates the first and second sets according to a PSM method described below.

In step 225, the processor 30 compares a first statistical distribution of the first set of PSM and a second statistical distribution of the second set of PSM to determine a likelihood of mTBI. For example, the likelihood of mTBI may be quantified as a percentage or a probability of occurrence of mTBI. In the exemplary embodiment, the first and second statistical distributions are compared using a Kullback-Liebler divergence (KLD) method or other statistical distribution methods. In particular, a large KLD indicates an increased likelihood of mTBI.

In a further embodiment, the likelihood of mTBI may be determined by generating a first KLD comparison and a second KLD comparison. The first KLD comparison is generated by selecting random samples of PSM generated from the stored data to create a set of random samples, as described above. Similarly, a statistical distribution of the set of random samples is generated by using KLD, thus generating the first KLD comparison. The first KLD comparison and second KLD comparison are then compared using the protocol described above. For example, to further accurately determine the likelihood of mTBI, generate a distribution of uninjured KLD comparisons by randomly taking samples of PSM measured during time epochs from the stored uninjured data and compare the random samples to the stored uninjured distribution of PSM values, and then compare the current distribution of KLD values to the uninjured distribution of KLD values to determine a likelihood of mTBI as a function of the KLD comparison.

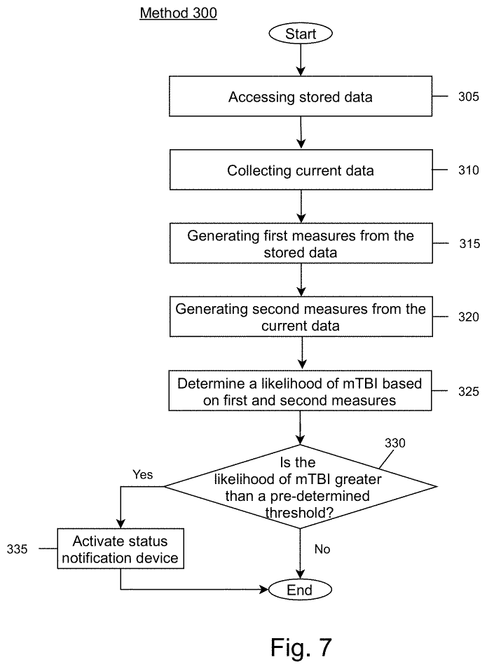

FIG. 7 shows an exemplary method 300 for detecting and alerting that the subject 80 is likely to have suffered from mTBI using the system 100. To notify that the subject 80 probably has the mTBI, the system 100 first determines the likelihood of the mTBI. Steps 305-325 for determining the likelihood of the mTBI are substantially similar to the steps 205-225 of method 200, as described above. For the purpose of describing the method 300, the method 300 will be applied to the subject 80 (e.g., a football player) as described above.

In step 330, the processor 30 determines whether the likelihood of the mTBI calculated in the step 325 is greater than a predetermined threshold, such as a predetermined threshold probability value. If in the step 330, it is determined that the likelihood of the mTBI is not greater than the predetermined threshold probability value, the method 300 moves to an end of the method and/or continues to monitor the subject 80 starting back at step 305. If in the step 330, it is determined that the likelihood of the mTBI is greater than the predetermined threshold probability value, the method 300 moves onto step 335. In one particular example, if the predetermined threshold probability value is 70% and the likelihood of the mTBI determined from the second data in the step 325 is 60%, the second data is moved to the memory arrangement 20 and stored, to be subsequently encoded as non-injured data. In another example, if the predetermined threshold probability value is 70% and the likelihood of the mTBI determined from the second data in the step 325 is 75%, the method 300 moves onto the step 335.

In the step 335, the transceiver arrangement 50 sends a message to the first computing unit 62 to activate the status notification device 64 of the head covering 60. The first transceiver arrangement 85 may receive the message, that is then transferred to the first processor 83. the first processor 83 may cause a change in voltage of the status notification device 64. In other words, the first processor 83 may either cause a voltage rise or a voltage drop in the status notification device 64 to activate the status notification device 64. For example, the voltage drop in the status notification device 64 causes the voltage dependent color changing paint on the head covering to change from a first color to a second color, signifying that the subject 80 likely has a mTBI and should be removed from the football game. In the example, the voltage dependent color changing paint may cover at least a portion of the head covering (e.g., a patch on the external surface of the head covering, or the entire external surface of the head covering). In another embodiment, the status notification device 64 may illuminate a LED or other light source on the subject, activate an alarm sound, send a haptic signal, and/or send an alarm signal to a remote monitoring device, among other possible notification. Further, the status notification device 64 may be powered by the portable power arrangement 68.

Detection of the mTBI is an estimation based on the electrical activity collected by the electrodes 74. The exemplary method 200 is based on five criteria. A first criteria is that there is no robust and unique, absolute measure of concussion available in an EEG, rather an impact-related change in the EEG can signal concussion. A second criteria is that there is no robust measure of concussive impact available from the accelerometers 76. A third criteria is that the concussion is identified by abnormal (e.g., decreased) long range synchrony between EEG channels, however there may be other signals as well. A fourth criteria is Normative EEG-derived data are unique for each subject, i.e., each of the subjects 80 will have a unique set of normative EEG-derived data. A fifth criteria is that normative EEG-derived data are unique for each activity the subject 80 may engage in. Additionally, there is a difficulty involved with collecting good

EEG data when the subject 80 is active, e.g., EEG data may be contaminated by artifacts of movement when the subject 80 is participating in a sport. During those specific moments of movement, as measured by the accelerometers 76, the EEG will be ignored because it will be contaminated by artifact.

As mentioned above, the impact protocol for determining whether the subject received an impact to the head is described as follows. The processor 30 receives the motion data collected by the accelerometers 76. The processor 30 then computes a head movement value by normalizing, summing, and/or averaging the motion data during configurable short epochs of time (e.g., at or about one-second). The processor 30 then computes a cumulative recent head impact (CRHI) value by summing large amplitude events from the acceleration output, sudden-onset of the head movement value, and short-lasting head acceleration signals, as a running average across the second time period (e.g., at or about two-hours), for example a user-specified time period, that depends on the activity of the subject during the second time period in which the likelihood of mTBI is being estimated. The sudden-onset of the head movement value may be determined when the head movement value exceeds a predetermined threshold value, such as 5 times a standard deviation of preceding or normative values. Specifically, the predetermined threshold value may be computed from the motion data collected when the subject 80 is substantially physically inactive (e.g., when the subject 80 is standing on the sidelines of a football game or otherwise at rest). An exemplary range of the short epochs of time may be between approximately a half-second to three-seconds. An exemplary range of the second time period may be between approximately a half-hour to four-hours.

In a further embodiment, the processor 30 may update stored PSM data with current PSM data after a user-defined sufficient period of time has passed during which the subject 80 has been deemed to have had no mTBI injury. The update allows the mTBI-determining method to be adaptive and increasingly informed, and accurate by the system 100 utilizing the method 200. For the PSM method mentioned above, the processor 30 generates the first and second sets of PSM from the first and second data, respectively. The PSM's are computed by a phase locking value (PLV), standard and debiased forms of the Weighted Phase Lag Index (WPLI, and dWPLI, respectively), coherence, as well as other computationally convenient methods that measure how a phase of one oscillating signal estimates a phase of another concurrent oscillating signal. The estimation is maximal (i.e., equal to 1) when a phase offset remains constant with a passage of time and the estimation is zero when the phase offset varies randomly in time.

A PSM of a pair of time-varying signals is computed in overlapping time windows of length that can vary to optimize computations. In the exemplary embodiment, the overlapping time windows are each four seconds in length and have a two-second overlap. The time-varying signals are first standardly analyzed for absence of extracerebral artifacts, then artifact-free EEG segments are band-pass filtered into distinct bands of interest. The bands of interest are theta (4-7 Hz), alpha (9-12 Hz), beta (15-25 Hz) and gamma (30-50 Hz) bands. The phases of the signals are extracted by narrow (e.g., at or about 3 Hz wide) band-pass filtering followed by a Hilbert transform. The band pass filtering may use an exemplary range of approximately 1 Hz to 25 Hz. In another embodiment, the bands of interests may be different from the bands mentioned above.

As mentioned above, the processor 30 runs the PSM method, which uses PLV, WPLI, dWPLI, and coherence, to generate the first and second sets of PSM from the first and second data. PSM is a vector comprised of one or more of the PLV, WPLI, or dWPLI measures but can include other measures like the spectral power within a frequency band such as 4-7 Hz theta, or other bands as defined above. The equation for calculating the PLV is as follows:

where t corresponds to time samples, j is the imaginary unit, T is the total length of the signal in samples, and corresponds to the instantaneous phase difference between the two signals. A second one of the plurality of equations is an equation for calculating the WPLI. The WPLI is based solely on the imaginary component of the cross-spectrum computed on short (e.g., 3-s) non-overlapping windows of narrow-band filtered data. The equation for calculating the WPLI is as follows:

where is the cross-spectrum of signals coming from a pair of electrodes, is the mean, is the imaginary component of the cross-spectrum and is its magnitude. The equation for calculating the dWPLI is as follows:

where, as above, is the cross-spectrum of signals coming from a pair of electrodes, is the mean, is the imaginary component of the cross-spectrum and is its magnitude. A fourth one of the plurality of equations is an equation for calculating the coherence. The equation for calculating the coherence is as follows:

where Gᵢⱼ(f) is the cross-spectral density between signals i and j, and Gᵢᵢ(f) and Gⱼⱼ(f) are the two autospectral densities of the i and j signals.

The mTBI-determining method is based on classification methods, similar to those used in machine learning, and other AI applications. The mTBI-determining method is personalized to the subject 80 and activity in which the subject 80 engages. Measures of frequency-band specific phase synchrony are computed between pairs of the electrodes 74 during time intervals that satisfy an inactivity criteria as measured by the accelerometers 76. If there is an “n” number of electrodes 74 then there are n(n−1)/2 pairs of the electrodes 74 and for each frequency band there will be a vector of dimension n(n−1)/2 that describes the band-specific phase synchrony across the EEG. Measurements can be made in k frequency bands such as approximately 0-4 Hz, 4-10 Hz, 10-15 Hz, 15-20 Hz. Assuming the k frequency bands, the vector of dimension will be kn(n−1)/2, thus describing the phase synchrony of the EEG at each measurement interval. The kn(n−1)/2-dimensional vector is in phase-synchrony state (PSS) because it describes the state of phase synchrony of the EEG at a moment. To simplify the computation only a subset of the kn(n−1)/2 measures that define PSS may be used. The subset is selected to be the most informative, which are theoretically identified and empirically validated measures of long-range phase synchrony. The mTBI-determining method however need not rely exclusive on these measures as additional measures such as estimates of phase-amplitude coupling may also prove to be useful, as well as other signals in the EEG.

In a further embodiment, the system 100 may be used with a person who does not have an uninjured baseline (i.e., no stored uninjured data because uninjured data has not been collected). The system 100 may instead use a normative distribution of EEG measurement data, for the person's demographic. In this embodiment, the system 100 may collect a first set of EEG data at a first time when the person is deemed to have a head injury (e.g., when the mTBI-determining method determines that the person has a concussion), collect a second set of EEG data at a second time after the first time, and compare the first set of EEG data to the second set of EEG data to determine whether the head injury was resolved at the time the second data were collected. Specifically, to determine whether the head injury was resolved, the second set of EEG data has a normative distribution (i.e., the normative distribution of EEG measurement data above), such that the first set of EEG data is compared to the normative distribution of the second set of EEG data. In other words, uninjured brain data and injured brain data used with the mTBI-determining method as described above may be substituted for normative brain data obtained using another method or technique.

### EXAMPLE

Described below is an example of the method of the present application using phase synchrony. It is noted that machine learning approaches typically do not rely on specific measures as the ones that are most informative get proportionally greater weight indicating their importance and those that contribute little to the intended discrimination get proportionally weaker weight indicating their unimportance.

To initialize and calibrate the system 100, the subject 80 wears the wearable garment 70 during uninjured baseline activities. The baseline activities may be similar to activities in which the subject 80 may possibly receive a concussion but need not be such activities. The wearable garment 70 records common conditions of the brain of the subject 80 during the baseline activities. The system 100 may collect EEGs during any of the common conditions, the common conditions defined by the action activities that the subject 80 is engaged in. The activities may be football, boxing, soccer, rest, reading, sleep, etc. EEG data collection during the common conditions allows the system 100 to collect and define a distribution of user- and activity-specific PSS measures that describe the common uninjured PSS. The distribution of user- and activity-specific PSS measures and/or statistics that describe the distribution may be stored on the memory arrangement 20. The distribution is comprehensive, whereas the statistics are a data and computationally compact approximation of the distribution. The distribution and the statistics may be a user- and activity-specific common, uninjured template PSS for subsequent concussion likelihood estimation.

During the concussion likelihood estimation, PSS measurements are collected during moments of inactivity, which may be verified by additional data collected by the accelerometers 76. The distribution PSS or an averaged PSS during a recent interval is computed as a current

PSS. The interval is a variable period of time (e.g., the last 30 s or 60 s, determined by the measurement conditions of the particular activity). At the interval, the current PSS is compared to the corresponding user- and activity-specific common template. The comparison is achieved by any number of statistically appropriate metrics, including Euclidean distance, Kullback-Liebler Divergence, z-score, bits, etc. The metrics describe deviation of the current PSS from the user- and activity-specific common template and is selected depending on a particular application.

Statistically large deviations of the current PSS from the common PSS increase the likelihood of a mTBI or a concussion. Deviations beyond a variable threshold, which is set to match a sensitivity that is appropriate for present circumstances of the subject 80, indicate a sufficiently high likelihood of concussion to trigger the status notification device 64. In another exemplary embodiment, the likelihood of concussion as a continuous variable may be used to continuously signal likelihood of concussion.

Concussion is a persistent injury that does not immediately heal, thus the method 200 computes a confidence and further likelihood of a concussion. False-alarms are inevitable in any detection system and the system 100 computes a user- and an activity-specific rate of false-alarms from data collected during construction of the user- and activity-specific common template. False alarms are statistical abnormalities; thus, they fluctuate rather than persist. On the other hand, true alarm of a concussion persistently exceeds the sensitivity threshold. The duration of time after an initial suprathreshold PSS event estimates the confidence in the identification of the concussion event. Thus, the system 100 estimates both the likelihood of concussion and the confidence in that likelihood estimate.

A concussion is unlikely to arise spontaneously as a concussion is likely to arise as a consequence of one or more accelerations of the head of the subject 80 being sufficient to generate substantial forces, especially shearing forces. The method 200 uses the acceleration of the head to estimate a likelihood of concussion. The threshold for the PSS deviation from the user- and activity-specific common template is a function of recent history of the accelerations of the head. Large recent accelerations of the head are measured and accumulated as an incidence metric using a leaky-integrator function where more recent events contribute more to a metric than more distant events. A time constant of an incidence accumulator is a variable that is set as appropriate for the particular activity the subject 80 is engaged in. For example, the time constant may be set for a duration of several football plays if the system 100 is used to estimate concussion during a football game or practice. The time constant may be set for an entire duration of a boxing match if the subject 80 is a boxer. The time constant may be set to several seconds (e.g., approximately 1-5 seconds) if it is used for detecting the likelihood of concussion due to a crash in a race car driver application. Regardless of the time constant, the incidence metric is used to adjust the threshold for setting the alarm to signal a concussion is likely. A greater incidence metric is set with a lower threshold.

The system 100 is designed to estimate concussion likelihood of the subject 80 on-the-field when the subject 80 participates in activities that may cause a concussion. In another exemplary embodiment, the system 100 may estimate concussion likelihood of the subject 80 off-the-field after the subject 80 performs the activities that may cause a concussion, such as when information increases a suspicion of the subject 80 having a concussion. When baseline, off-the-field EEG is collected by the wearable garment 70, the PSS measurements define the user- and activity-specific common template. For example, the activities may be one of resting, reading, sleeping, being in an ice-bath, participating in a standardized cognitive testing, or other participating in a concussion assessment effort, etc. The subject 80 may engage in any of the previously mentioned activities and any of the previously mentioned activities may be used as a baseline for estimating functional brain state. The mTBI-determining method operates in the same manner as described above, i.e., comparing current PSS to common distribution of PSS.

The system 100 may be used in combination with anti-concussion efforts, both during the on-the-field activities with substantial danger of causing concussion and off-the-field activities when a concussion is unlikely to be caused but likely to be present if the concussion was caused during the on-the-field activities. The robustness is achieved because with the combined use of the system 100 providing multiple tiers of protection against missing the signals and opportunity to detect concussion.

The system 100 may be a consumer device or a medical device, in each case the design specifications will differ as necessary to meet application demands. In the exemplary embodiment, the system 100 is a consumer device that is operated independently of medical personnel, the consumer device being in a category of consumer devices including a home thermometer, a blood-pressure monitor, and a glucose monitor. The system 100 may be used to indicate to the subject 80 or a consumer user that there is an objective reason for the subject 80 to see a physician. In this consumer embodiment it is anticipated that the mTBI-determining method operates in the same manner as described above, i.e., comparing the current PSS to a common normative distribution of PSS data obtained using another method or technique.

As mentioned above, the accelerometers 76 measure acceleration of the head of the subject 80 in multiple dimensions. In the exemplary embodiment, the wearable garment 70 has three separate accelerometers 76 to estimate the acceleration of the head of the subject 80. The acceleration is produced by shearing forces. Accordingly, the acceleration produces large coplanar accelerations in a particular plane across the head of the subject 80. Planar components of the head accelerations may be estimated by summing simultaneous standard trigonometric component projections from different accelerometers 76. A shearing head acceleration has most of the acceleration in a particular plane. Head accelerations with strong shearing features are noted and used to adjust the suspicion of concussion and lower the threshold for concussion detection using a leaky-integration method described below.

As mentioned above, the wearable garment 70 must be placed onto the head of the subject 80 in the desired configuration such that the electrodes 74 are reliably positioned on the head. In an exemplary embodiment, the wearable garment 70 may have automatic registration. The automatic registration automatically identifies signals from each of the electrodes 74 when the wearable garment 70 is placed in the desired configuration. The wearable garment 70 thus places the electrodes 74 in discrete geographical locations in a stereotyped arrangement around the head, however the wearable garment 70 may be rotated on the head. In an exemplary embodiment, the wearable garment 70 may have guiding markers to guide placement of the wearable garment 70 in the desired configuration. Registering positions of the electrodes 74 on the head of the subject 80 may be done by an EEG technician or self-applied by the subject 80 using the automatic registration of the wearable garment 70. For example, when only the transverse band 75 is used without the sagittal band 73 or the coronal band 71, the registration may be done automatically to register the electrodes 74.

In a further exemplary embodiment, the electrodes 74 may have a registration process for positions and identifications of the electrode 74. For example, applications with the head covering 60 may be used as a standard coordinate system for registration of the electrodes 74. Accordingly, a pair of unique electrical signals may be provided at an underside of the head covering 60 close to a scalp of the subject 80. During the registration process, the unique electrical signals are generated between a pair of two closely spaced signal generating electrode poles. The signal generating electrode poles are mounted on the underside of the head covering 60, on either side of the head of the subject 80. The wearable garment 70 may pick up the signals and the processor 63 measures amplitudes of the unique electrical signals of the electrode 74 and thereby localize the amplitudes because a largest amplitude signal is from the electrode 74 that is closest to the electrode poles. In an exemplary embodiment where the head covering 60 is not used, ears of the subject 80 may be used to establish the standard coordinate system. Accordingly, the signal generating electrode poles may be sized and shaped to conform to the ears of the subject 80. The subject 80 may use the signal generating electrode poles with the wearable garment 70.

In collecting the brain function data upon which a measure of mTBI can be based, the data when combined with information about consumer activities, can also subsequently be used to make appropriate recommendations for future consumer activities that are based on assessments of recent and/or current brain function. Like with most injuries, it is important to identify mTBI early to improve the chances of a good outcome by managing the condition, nonetheless as with most injuries mTBI is rare, and the user of the present art will most of the time be collecting brain function data during normal activities that are not associated with injury. The statistics of the normal, non-injured data are stored and used for the algorithmic detection of injury-associated brain function, but the data are also useful for other purposes.

If the user elects to use a smart phone or similar device to register their mood, social activities, significant communications such as phone calls, or their commercial activities such as shopping, entertainment, outings to restaurants and other events, then these behavioral choices can be associated with the brain function data. It is possible to use brain function patterns in the data to predict prospective behaviors using standard machine learning techniques. In this case, the present art can also be used to make prospective recommendations of desirable activities (e.g., “call your mother”, “consider eating out tonight”) or prospective warnings (e.g., “you might not want to shop today”, “you might consider taking a break from social media this afternoon”) or retrospective queries (e.g., “seems like something is up, did something recently upset you?”). These actions would be based on finding a specific user's association between brain function patterns and the registered events and behaviors. One version of such a system, like the economic model of search and social media, could make an online marketplace aware of what was assessed to be what a user might be interested in accessing. This could be a vacation, a restaurant reservation, or a physical activity, etc. The present art could then also serve to direct personalized, brain function-based marketing and search, in addition to detecting mTBI.

The invention described and claimed herein is not to be limited in scope by the specific embodiments herein disclosed since these embodiments are intended as illustrations of several aspects of this invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. All publications cited herein are incorporated by reference in their entirety.

1. A system for detecting mild traumatic brain injury (mTBI), comprising:
a memory arrangement including stored brain data corresponding to an electrical activity of a brain of a subject at a plurality of locations in the brain during a first time period; and
a processor receiving: (i) the stored brain data and (ii) current brain data corresponding to the electrical activity of the brain of the subject at the plurality of locations during a second time period, wherein the second time period is after the first time period, the processor generating a first set of phase synchrony measures (PSM) corresponding to frequency band-specific oscillatory phase synchrony of the stored brain data, and a second set of PSM corresponding to frequency band-specific oscillatory phase synchrony of the current brain data, the processor determining a likelihood of mTBI based on the first and second sets of PSM.

2. The system of claim 1, wherein the first time period corresponds to a time period during which the subject is substantially physically inactive.

3. The system of claim 1, wherein the second time period corresponds to a time period during which the subject is engaged in physical activity.

4. The system of claim 1, wherein the processor is further configured to generate a first statistical distribution of the first set of PSM and a second statistical distribution of the second set of PSM and compare the first and second statistical distributions to determine the likelihood of mTBI.

5. The system of claim 1, wherein the processor further receives (iii) motion data corresponding to an acceleration motion of a head of the subject during the second time period and wherein the processor is further configured to generate a cumulative recent head impact (CRHI) value based on the motion data, and to generate the second set of PSM using only those portions of the current brain data corresponding to time epochs within the second time period when the CRHI value exceeds a predetermined threshold value.

6. The system of claim 5, wherein the CRHI value is generated based on absolute magnitudes of the acceleration motion of the head of the subject.

7. The system of claim 5, wherein the predetermined threshold value corresponds to the acceleration motion of the head when the subject is substantially physically inactive.

8. The system of claim 1, further comprising:
a plurality of electrodes coupled to a wearable garment for a head of the subject so that, when the garment is worn in a desired configuration, each of the electrodes is positioned on a portion of scalp adjacent to a corresponding one of the plurality of locations, the electrodes being couplable to the processor to transfer data corresponding to the detected electrical activity thereto.

11. The system of claim 8, wherein a portion of the plurality of electrodes is positioned along a transverse band of the head of the subject, the transverse band approximating a circumference of the head of the subject.

10. The system of claim 8, wherein a portion of the plurality of electrodes is positioned along at least one of a coronal band of the head of the subject and a sagittal band of the head of the subject.

11. The system of claim 8, further comprising:
at least one accelerometer coupled to the garment, the at least one accelerometer being couplable to the processor to transfer data corresponding to the detected acceleration motion of the head of the subject.

12. The system of claim 11, wherein the at least one accelerometer is configured to detect linear acceleration in vertical and horizontal planes, or rotational acceleration in pitch, yaw, and roll.

13. The system of claim 11, further comprising:
a data communications device operably connected to the plurality of electrodes and the at least one accelerometer so that the electrical activity detected by the electrodes and the acceleration motion detected by the at least one accelerometer are communicated via the data communications device to the processor.

14. The system of claim 13, wherein the data communications device communicates with the processor via a wired or wireless connection.

15. The system of claim 1, wherein the likelihood of mTBI is a probability that the subject suffered from mTBI, and the processor is further configured to activate an external signal when the probability that the subject suffered from mTBI is above a predetermined threshold probability value.

16. The system of claim 15, further comprising:
a helmet placed over a wearable garment onto the head of the subject,
wherein the external signal is at least one of changing a color of a whole or a portion of the helmet, illuminating a LED or other light source on the subject, activating an alarm sound, sending a haptic signal, and sending an alarm signal to a remote monitoring device.

17. The system of claim 1, wherein the stored brain data corresponds to a normative distribution of data of the electrical activity of the brain determined from a set of standards.

18. A method for detecting mild traumatic brain injury (mTBI), comprising:
accessing stored data corresponding to an electrical activity of a brain of a subject at a plurality of locations in the brain during a first time period;
collecting current data corresponding to the electrical activity of the brain of the subject at the plurality of locations during a second time period, wherein the second time period is after the first time period;
generating a first set of phase synchrony measures (PSM) corresponding to frequency band-specific oscillatory phase synchrony of the stored data, and a second set of PSM corresponding to frequency band-specific oscillatory phase synchrony of the current brain data; and
determining a likelihood of mTBI based on the first and second sets of PSM.

19. The method of claim 18, wherein the first and second sets of PSM are compared using a Kullback-Liebler divergence (KLD) method to determine the likelihood of mTBI.

20. The method of claim 18, wherein the first time period corresponds to a time period during which the subject is substantially physically inactive.

21. The method of claim 18, wherein the second time period corresponds to a time period during which the subject is engaged in physical activity.

22. The method of claim 18, wherein the determining step comprises:
generating a first statistical distribution of the first set of PSM and a second statistical distribution of the second set of PSM; and
comparing the first and second statistical distributions to determine the likelihood of mTBI.

23. The method of claim 18, further comprising:
receiving motion data corresponding to an acceleration motion of a head of the subject during the second time period; and
generating a cumulative recent head impact (CRHI) value based on the motion data, and wherein the second set of PSM is generated using only those portions of the current brain data corresponding to time epochs within the second time period when CRHI value exceeds a predetermined threshold value.

24. The method of claim 23, wherein the predetermined threshold value corresponds to the acceleration motion of the head when the subject is substantially physically inactive.

25. The method of claim 23, wherein the time epochs each have a duration of about one second.

26. The method of claim 18, wherein the second time period has a duration of about two hours.

27. The method of claim 18, further comprising:
activating an external signal when a probability that the subject suffered from mTBI is above a predetermined threshold probability value, wherein the external signal is at least one of changing a color of a whole or a portion of a helmet, illuminating a LED or other light source on the subject, activating an alarm sound, sending a haptic signal, and sending an alarm signal to a remote monitoring device.
